(19) United States
(12) Patent Application Publication
(10) Pub. No.: US 2026/0221244 A1
(43) Pub. Date: Jul. 30, 2026

(54) RESOURCE-EFFICIENT MANAGEMENT OF PATIENT CONSENT AT A DEVICE LEVEL

(71) Applicant: Alcon Inc., Fribourg (CH)

(72) Inventors: Thomas E. Jackson, Laguna Niguel, CA (US); Nikita Mishra, Foothill Ranch, CA (US)

(21) Appl. No.: 19/451,220

(22) Filed: Jan. 16, 2026

Related U.S. Application Data
(60) Provisional application No. 63/750,069, filed on Jan. 27, 2025.

(51) Int. Cl. G16H 10/60

(52) U.S. Cl. CPC G16H 10/60

(57) ABSTRACT

A computer-implemented method of resource-efficient consent management. Embodiments include receiving, at a device, an indication that consent has been given with respect to one or more items. Embodiments include determining, at the device, a binary bit pattern representative of the indication based on a configured association between the one or more items and one or more values in the binary bit pattern. Embodiments include storing, at the device, a memorialization of the indication via a bit vector having the binary bit pattern.

### CROSS-REFERENCE TO RELATED APPLICATIONS

This application claims the benefit of U.S. Provisional Application Ser. No. 63/750,069 (filed on Jan. 27, 2025), the content of which is incorporated by reference herein in its entirety.

### BACKGROUND

Managing memorializations of consent related to functionality of a device, such as a medical device, presents certain challenges. Consent management is essential during clinical trials, patient diagnosis, surgeries, and many other contexts. Currently, consent associated with medical devices is manually managed, resulting in significant paperwork, effort, and inefficiency. Managing and logging changes to preferences is also a challenging effort with existing manual approaches. Furthermore, manual consent management can also be the source of errors.

It may be advantageous in many cases to store and manage records of consent having been given (e.g., by patients) to particular items such as medical procedures or processes related to functionality of a device (e.g., a device used in connection with such procedures or processes) on the device itself. Storing and managing records of such consent may be performed in order to comply with applicable laws, regulations, and/or the like, and may often involve granular consent being given or not given with respect to a potentially large number of items. Often, however, resource constraints exist on devices due to factors such as physical size and the importance of optimizing for power, cost, consumption, and overall efficiency. For example, in the context of software in a medical device (SiMD) there are often limitations in memory, processing power, power consumption, storage space, network bandwidth, input/output (I/O) capabilities, environmental conditions, and/or the like.

Accordingly, there is a need for any consent management solution at the device level to be highly efficient in terms of memory, processing power, and storage.

### SUMMARY

In certain embodiments, one general aspect includes a computer-implemented method for resource-efficient consent management. The computer-implemented method includes: receiving, at a device, an indication that consent has been given with respect to one or more items; determining, at the device, a binary bit pattern representative of the indication based on a configured association between the one or more items and one or more values in the binary bit pattern; and storing, at the device, a memorialization of the indication via a bit vector having the binary bit pattern.

In certain embodiments, another general aspect includes a system. The system includes a memory having executable instructions and a processor in communication with the memory. The processor is configured to execute the instructions to perform the computer-implemented method for resource-efficient consent management described above.

In certain embodiments, another general aspect includes a computer-program product including a non-transitory computer-usable medium having computer-readable program code embodied therein. The computer-readable program code is adapted to be executed to implement the computer-implemented method for resource-efficient consent management described above.

### BRIEF DESCRIPTION OF THE DRAWINGS

FIG. 1 illustrates an example of a computing environment for resource-efficient consent management, in accordance with certain embodiments of the present disclosure.

FIGS. 2A, 2B, and 2C illustrate examples related to a bit vector for resource-efficient consent management, in accordance with certain embodiments of the present disclosure.

FIG. 3 illustrates an example of storage formats for resource-efficient consent management, in accordance with certain embodiments of the present disclosure.

FIG. 4 illustrates an example of a computing device for resource-efficient consent management, in accordance with certain embodiments of the present disclosure.

FIG. 5 illustrates an example of a process for resource-efficient consent management, in accordance with certain embodiments of the present disclosure.

### DETAILED DESCRIPTION

Consent management at the device level in connection with procedures or operations performed by devices (e.g., medical devices) is challenging due to resource constraints of such devices. Techniques described herein enable resource-efficient storage and management of consent at the device level through the use of bit vectors, optimized associations between bit patterns and item-level consent, and dynamic access and synchronization solutions for such consent data.

In some embodiments, a bit vector is used to memorialize consent with respect to a variety of different items. A bit vector is a mutable sequence of Boolean values, and may be used a compact representation of consent data. For example, a bit vector may be an n-bit wide integer (e.g., 24-bit, 32-bit, 64-bit, and/or the like) that can represent n distinct and discrete values or 2\hat{ }n different values. In one embodiment, each bit in the bit vector may be associated with a particular item to which consent may be given, with a one value indicating that consent has been given to the item or a zero value indicating that consent has not been given to the item. In other embodiments, multiple items may be associated with a single bit in the vector, such that a one value for the bit indicates that consent has been given to the multiple items. In additional embodiments, certain bit patterns may be associated with particular sets of items, such as an association indicating that if the count of one values up to a particular offset in the bit vector is equal to a certain number then a particular set of items have been consented to.

Metadata associated with consent information may also be stored in one or more bit vectors, such as a separate bit vector from the bot vector used to store the consent information itself. Such metadata may include timestamp information (e.g., indicating when consent was received for a given item), location information, identifier(s), and/or the like.

Bit vectors storing consent information as described herein may be efficiently queried and/or otherwise processed to determine whether consent has been given to particular items through configured functions that enable retrieval of a particular bit value (e.g., at a given offset relative to a least significant bit), retrieval of a count of one values or zero values up to a particular offset, retrieval of an offset up to which a given count of one values or zero values is present, and/or the like.

Furthermore, bit vectors storing consent information and/or related metadata as described herein may be synchronized between a device and a separate entity such as a cloud server in order to maintain consistent consent management, such as across multiple devices. In some cases, an event loop on a device may monitor for situations in which new consent information is needed, obtain such consent information (e.g., via one or more I/O components), set bit vectors accordingly, and/or transmit bit vectors to a cloud server when certain trigger conditions are met (e.g., expiration of a timer, creation or modification of a bit vector, amount of data being stored on the device, certain input being received via a user interface, shutdown of the device, upon a push event from a cloud server, and/or the like). In some embodiments, the cloud server may synchronize consent information received from one device with one or more other devices that utilize such consent information, such as a software as a medical device (SaMD) component.

Embodiments of the present disclosure accomplish various technical improvements. For example, utilizing bit vectors and configured associations between particular items to which consent may be given and particular bits or bit patterns enables storage and management of consent at the device level in a manner that makes efficient use of memory, processing, and/or communication resources, thereby enabling storage and management of such information even on devices with limited computing resources. Accordingly, techniques described herein improve the functioning of computing devices involved (e.g., by utilizing fewer computing resources than alternative consent management techniques, such as storing consent information in larger, more resource-intensive forms) and enable computer-based consent management in cases where such functionality would not have previously been feasible from a resource perspective at the device level. Furthermore, functions described herein for retrieving a particular bit value at a given offset, a count of one values or zero values up to a particular offset, an offset up to which a given count of one values or zero values is present, and/or the like improve efficiency of querying the consent data and reduce the time it takes to access, count, and select the individual bits, thereby further improving the functioning of the computing device(s) involved and the technical field of computer-based consent management. Additionally, embodiments described herein involve automated logic for obtaining consent information, storing obtained consent information (and, in some embodiments, associated metadata) in resource-efficient bit vectors, and/or synchronizing such consent information across multiple devices for improved consent data accessibility, consistency, and management, avoiding errors that may be introduced through manual consent management solutions and enabling dynamic and optimized computer-based consent management across a device ecosystem.

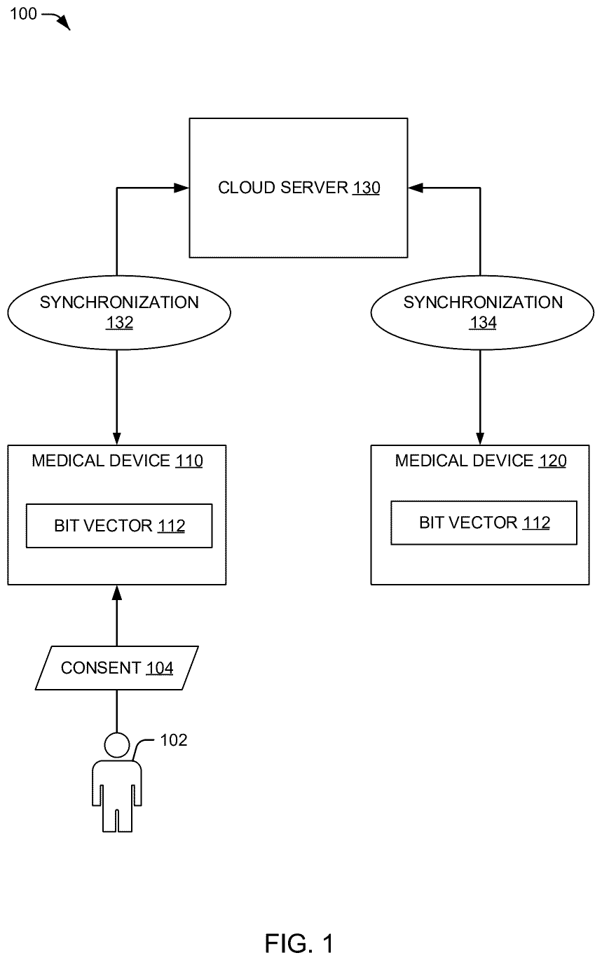

FIG. 1 illustrates an example of a computing environment 100 for resource-efficient consent management, in accordance with certain embodiments of the present disclosure.

In computing environment 100, medical devices 110 and 120 are connected to a cloud server 130, such as via a network connection (e.g., the Internet or any other connection over which data may be transmitted). Medical devices 110 and 120 generally represent devices that are used in connection with performing one or more medical procedures or processes. For example, medical device 110 or 120 may comprise software in a medical device (SiMD) and/or software as a medical device (SaMD) components. It is noted that while certain embodiments are described with respect to medical devices, techniques described herein may also be used with other types of devices (e.g., any type of device comprising storage resources with which consent information may be stored).

In one particular example, medical device 110 is an ophthalmic imaging device that has an ophthalmic imaging software application resident and executing thereon. For example, medical device 110 may be operable to capture and/or process images of an eye of a patient, and to store such images in relation to the patient.

In various embodiments, medical device 110 is configured to obtain consent 104 from the patient (e.g., user 102 may represent the patient or a user of medical device 110 that is associated with the patient and provides input indicating that the patient has provided consent) to one or more items, such as aspects related to one or more medical procedures and/or processes. For example, medical device 110 may prompt user 102 to provide input indicating consent to one or more items, such as via a user interface provided via a display, audio, and/or the like. In one example, such a prompt is provided via a graphical user interface (GUI). User 102 may input consent 104, such as indicating whether the patient consents to each of one or more items, via such a user interface, such as providing input via one or more input devices (e.g., audio input, touch input, keyboard and/or mouse input, button input, and/or other type of interaction with one or more components). Consent 104 generally indicates whether the patient consents to one or more items, such as preferences, procedures, processes, operations, and/or the like. Examples of items to which consent may be given and to which consent 104 may relate include allowing the storage of preferences for a given amount of time, allowing the storage of diagnostic images for a given amount of time, allowing the storage of personally identifiable information (PII) and/or personal health information (PHI) in a given geographic region, using diagnostic images for data analysis and/or process improvement, using personal characteristics to determine a probability of a disease in the future, using local or general anesthesia for a procedure, prioritizing a device's intelligence above a doctor's recommendation for a medical-related determination such as selection of an eye lens, and/or the like.

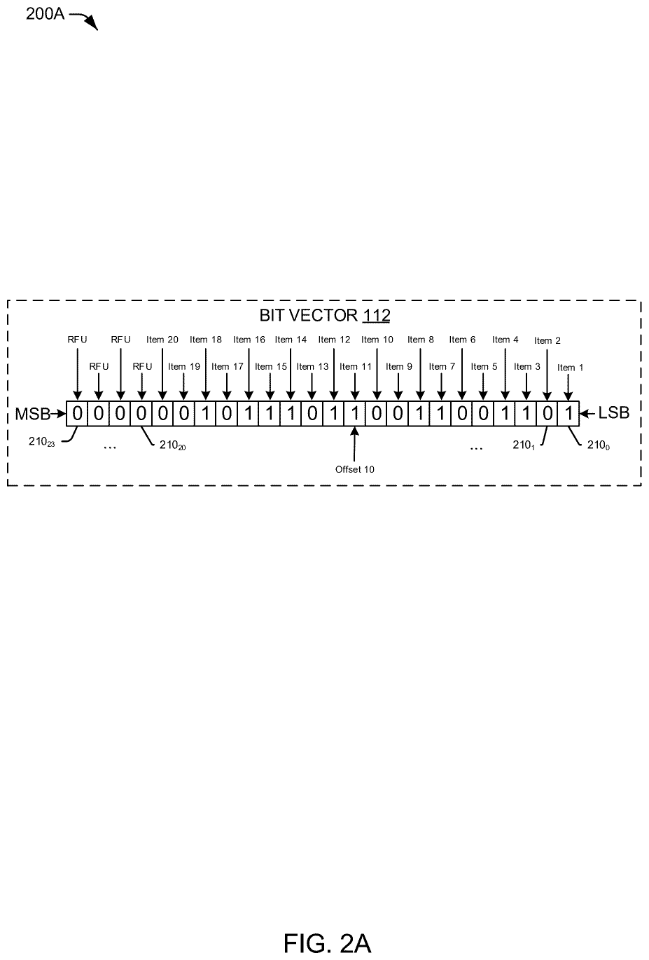

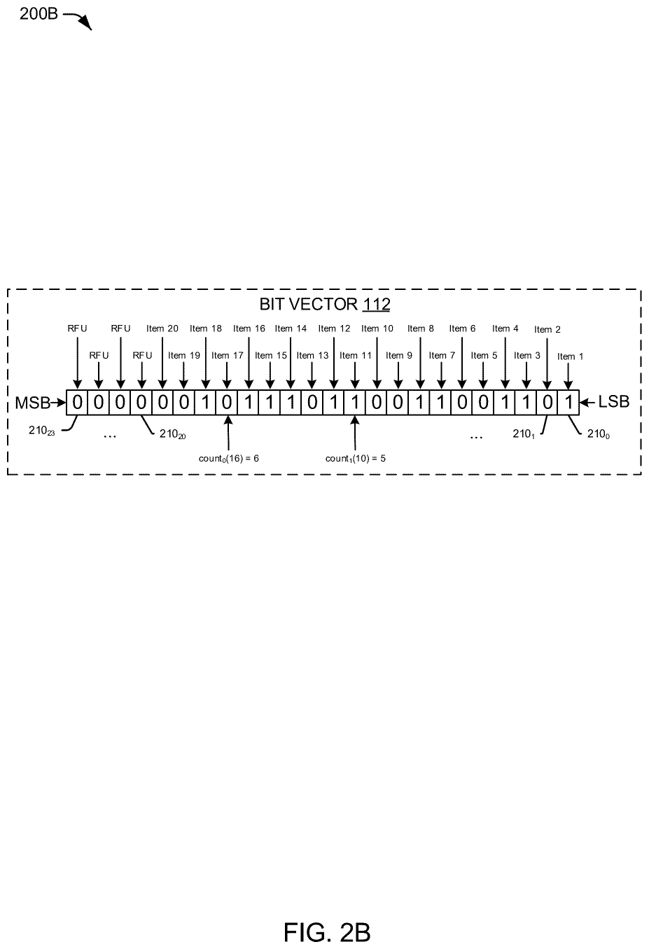

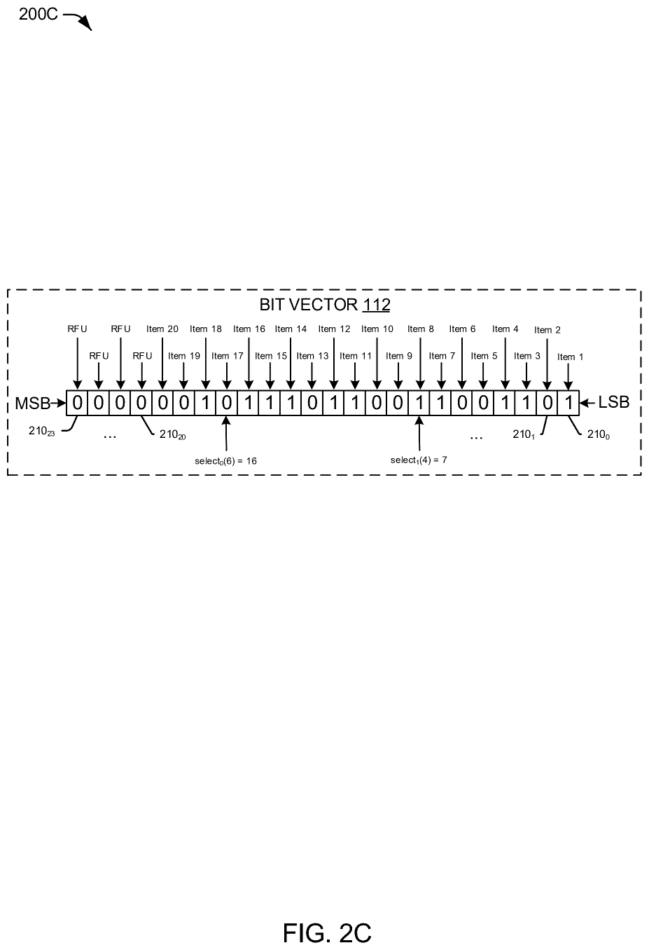

Medical device 110 may memorialize consent 104 in the form of a bit vector 112, which comprises a resource-efficient representation of whether the patient consents to each of a plurality of items. Examples of bit vector 112 are described in more detail below with respect to FIGS. 2A, 2B, and 2C. For example, bit vector 112 may be an n-bit integer comprising a series of n Boolean values in a pattern that indicates consent 104 and/or other consent information (e.g., received independently of consent 104). In some embodiments each bit of bit vector is associated with a particular item, while in other embodiments a single bit in bit vector 112 is associated with multiple items. In certain embodiments particular bit patterns are associated with particular combinations of items. For example, a certain count of one or zeros up to a particular offset (e.g., from a least significant bit of bit vector 112 up to that offset) may be mapped to a particular combination of items such that using a bit pattern in bit vector 112 that includes such a count of ones or zeros up to that particular offset indicates that the patient has consented to that particular combination of items. Such association(s) may be stored in logic on medical device 110 such that medical device 110 is able to generate bit vector 112 based on consent information received from one or more users.

In some embodiments, an event loop runs on medical device 110 that monitors when new consent preferences are needed, obtains such consent preferences (e.g., prompting a user to provide consent 104), and sets the bit vectors accordingly (e.g., bit vector 112 and/or one or more other bit vectors, such as a bit vector storing metadata associated with consent 104). Such an event loop may be protected by a watchdog mechanism that ensures the service is running and restarts the service if necessary (e.g., if the watchdog mechanism detects that the service is not running). The event loop may also securely transit the bit vector(s) to cloud server 130, such as using transport layer security (TLS) or another secure transmission technique, based on occurrence of one or more pre-defined triggers (e.g., each time bit a bit vector is created or modified, at regular intervals, whenever a certain amount of consent data has been stored on medical device 110, when certain input is received via a GUI, upon shutdown of medical device 110, upon a push event from cloud server 130, and/or the like). For example, at synchronization 132, bit vector 112 (and, in some embodiments, an associated bit vector storing metadata) is transmitted to cloud server 130, such as upon occurrence of a trigger condition.

In some cases, at synchronization 134, cloud server 130 also synchronizes consent data with one or more other devices such as medical device 120. For example, medical device 120 may be a different device than medical device 110, and may have use of the consent data in connection with one or more medical procedures or processes. Cloud server 130 may transmit bit vector 112 to medical device 120 at synchronization 134, such as upon receiving bit vector 112 from medical device 110 at synchronization 132 and/or upon the occurrence of one or more other trigger conditions. Cloud server 130 may also transmit one or more other bit vectors (e.g., an associated bit vector storing metadata) along with bit vector 112 to medical device 120 at synchronization 134. Medical device 120 may utilize bit vector 112 in connection with performing one or more operations, such as to confirm consent to one or more items. In one embodiment, medical device 120 comprises a software as a medical device (SaMD) component running within a software in a medical device (SiMD) component on a medical device.

In some embodiments, synchronization 132 and/or 134 involves synchronizing a subset of the consent data that is relevant to one or more particular devices. For example, if not all items for which consent data is available are relevant to a particular device associated with a patient, then only consent data for relevant items to the particular device may be synchronized with the particular device while consent data for irrelevant items may not be synchronized with the particular device. The access, count, and/or select functions may be involved in the synchronization process

In some embodiments, medical devices (e.g., medical device 110 and/or 120) may use progressive web applications (PWAs), which are standard web applications existing offline like a desktop application and caching data to sync up with one or more server(s) (e.g., cloud server 130) once online. In some embodiments, medical devices can store preferences relevant to an SaMD or other component on the device using a separate bit vector to reduce effective storage space required for those preferences.

In certain embodiments metadata associated with consent 104 is stored in a separate bit vector from bit vector 112. For example, such an additional bit vector may be utilized to capture relevant metadata for the consent such as timestamping information, a location (e.g., geographic location) of a health delivery organization (HDO), an identifier of an HDO, a case identification (case ID), and/or the like. In some cases, a case ID in combination with the timestamping and location metadata may be used to accurately identify a patient case. For example, an n-bit vector may be divided into sections (e.g., 8-bit sections, sections of varied sizes, and/or the like) that represent different metadata values, such as a first section being associated with a timestamp (e.g., represented as an offset from coordinated universal time (UTC)), a second section being associated with a geographic location (e.g., represented as physical coordinates, a location identifier, an HDO identifier, and/or the like), a third section being associated with an identifier (e.g., represented as an integer or other series of digits), and/or the like. Such associations may be configured in advance and used to generate a metadata bit vector associated with bit vector 112.

In some embodiments, a technique for alleviating the burden on the medical device includes post processing of the consent data. For example, once a set of one or more bit vectors is uploaded to a cloud component (e.g., cloud server 130), such bit vectors may be matched and parsed to a configured dictionary (e.g., stored on cloud server 130) that describes exactly what the contents of the bit vectors represent (e.g., associations between items and bits, bit patterns, bit sections, and/or the like, associations between metadata items and bits, bit patterns, bit sections, and/or the like).

Furthermore, a list of HDOs along with associated locations may be stored (e.g., on cloud server 130) and matched by using an HDO location or identifier that is set in a bit vector. Such an approach allows the medical device to set and upload the consent management information in a resource-efficient manner while still enabling such information to be automatically processed and understood (e.g., at cloud server 130). Capturing the geographic location for an HDO enables consenting to privacy regulations across the world in such a way that an organization or entity can ensure that when collecting consent, it is complying with consent laws or regulations within the applicable jurisdiction(s).

Bit vector 112 (and/or one or more other bit vectors, such as storing associated metadata and/or other preferences) may be efficiently queried at a given endpoint (e.g., cloud server 130, medical device 110, and/or medical device 120) in order to retrieve data, such as via one or more functions. In one example, an access function, a counting function, and a selecting function are provided.

An access function may accept an offset index as an input and may return a Boolean value from that offset index in the bit vector as an output. In one embodiment, such an access function is represented as CM·access(i)=CM·[i], where CM refers to a consent management (CM) bit vector and i refers to an offset index. For example, given an offset the access function may return the value of whether the bit at the offset is set within the bit vector or not. The offset index range may start at zero, which may correspond to the least significant bit (LSB) of the bit vector.

A counting function may provide an efficient way to count the discrete ones or zeros in the bit vector up to, but not including, a given offset index. For instance, a count ones function may accept an offset index as an input, and may return a count of one values up to, but not including the offset index and a count zeros function may accept an offset index as an input, and may return a count of zero values up to, but not including the offset index. In one embodiment, a count ones function is represented as

$$CM \cdot {count}_{1} ( i ) = {\sum }_{j = 0}^{i - 1} CM [ j ] ,$$

where CM refers to a consent management (CM) bit vector, i refers to an offset index, j refers to a counter variable for a summation. For example, given an offset the count ones function may return the sum of the values from the bit vector at each index from index zero (e.g., the LSB) up to, but not including, the offset (e.g., up to the offset minus one). In one embodiment, a count zeros function is represented as CM·count₀(i)=i−CM·count₁(i), where CM refers to a consent management (CM) bit vector, i refers to an offset index, and count₁ refers to the count ones function discussed previously. For example, given an offset the count zeros function may return the difference between the offset itself and the count of ones up to, but not including the offset. This difference will be equal to the count of zeros up to, but not including the offset. For example, given offset i=5, if there are three ones up to, but not including, offset index 5, then count₁(5)=3 and count₀(5)=5−3=2 (because if 3 of the 5 bits prior to offset 5 are equal to 1, then the other 2 of the 5 bits prior to offset 5 must be equal to 0).

A select function may provide an efficient way to determine the offset index such that the count of ones or zeros up to, but not including, the offset index equals a given value. For instance, a select ones function may accept a count value (e.g., an integer) as an input, and may return an offset index for which the count of one values up to, but including the offset index equals the count value and a count zeros function may accept a count value (e.g., an integer) as an input, and may return an offset index for which the count of zero values up to, but including the offset index equals the count value. In one embodiment, a select ones function is represented as CM·select₁(i)=max {j|CM·count₁(j)=i}, where CM refers to a consent management (CM) bit vector, i refers to a count value, j refers to an offset index, and count₁ refers to the count ones function discussed previously. For example, CM·select₁(i) may return a maximum j such that count₁(j)=i. In one embodiment, a select zeros function is represented as CM·select₀(i)=max {j|CM·count₀(j)=i}, where CM refers to a consent management (CM) bit vector, i refers to a count value, j refers to an offset index, and count₀ refers to the count zeros function discussed previously. For example, CM·select₀(i) may return a maximum j such that count₀(j)=i. An example related to CM·select₁(i) is described below with respect to FIG. 2C.

Such functions may be part of a data structure of the bit vector, and may be called in order to invoke applicable functionality. Each of these functions, access, counting, and selecting, operate in constant time, also known as O(1) in Big O Notation. Thus, the running time of such functions is independent of the size of the input. One reason for increased time efficiency of such functions is that the algorithms are essentially operating as lookup tables. The execution time remains unchanged irrespective of the input. Additionally, the size of a bit vector is pre-defined and does not grow.

According to techniques described herein, a connected medical device can enable consent management to be recognized at the device level. For example, a connected medical device may be a pre-operative diagnostic device or a surgical device console. Although described with respect to a connected device, techniques described herein may be utilized in a stand-alone or disconnected device also. Embodiments of the present disclosure enable a patient's privacy and consent preferences to be collected and shared in compliance with applicable regulations, laws, and/or requirements, beginning at the medical device itself.

In some cases, a medical device or system can be overly stringent about collecting consent information, such as requiring acceptance of consent to a list of items before enabling use of the device. In many cases, it is not possible to use a device without agreeing to such statements asking for consent. In such cases (and others), embodiments of the present disclosure may enable efficient processing of consent revocation. For example, with resource efficient synchronized bit vectors as described herein, revocation of consent may be accomplished and synchronized across devices more easily, making such functionality more maintainable across devices. For example, if a patient revokes consent to one or more items after initially providing consent (e.g., to enable use of the device), such revocation may be efficiently processed through modification to one or more bit vectors and efficiently synchronized across devices via such modified bit vector(s).

FIGS. 2A, 2B, and 2C illustrate examples 200A, 200B, and 200C related to a bit vector for resource-efficient consent management, in accordance with certain embodiments of the present disclosure. Examples 200A, 200B, and 200C include bit vector 112 of FIG. 1.

In each of examples 200A, 200B, and 200C, bit vector 112 is a 24-bit vector including a plurality of bits 210₀₋₂₃, where the value at a given bit 210ₙ is a Boolean value of either 0 or 1. For instance, bit 210₀ refers to the bit at offset index 0 and has a Boolean value of 1 (e.g., meaning that the bit is set), while bit 210₁ refers to the bit at offset index 1 and has a Boolean value of 0 (e.g., meaning that the bit is not set). Offset 10 refers to the 11th bit starting from the least significant bit (LSB) (e.g., the LSB has an offset value of 0). The LSB is 210₀ while the most significant bit (MSB) is 210₂₃.

In one or more of the depicted examples, each bit 200ₙ is associated with a particular item (e.g., item 1, item 2, . . . item 20) or is reserved for future use (RFU). For example, bit 200₀ is associated with Item 1, meaning that if bit 200₀ is set (e.g., having a value equal to 1) then this indicates that consent has been given to Item 1 (e.g., which may refer to any given item to which consent may be given, such as a preference, procedure, operation, process, and/or the like). Bits that are reserved for future use (e.g., bits 200₂₀₋₂₃) may refer to bits that have not yet been associated with particular items, and that can be associated with items at a later time (e.g., if new items are identified for inclusion in the consent management system). The ability to have reserved space (e.g., bits that are RFU) is advantageous for being able to adapt to changing consent management requirements. For example, such embodiments are flexible enough to allow for future expansion and requirements.

In alternative embodiments, multiple items may be associated with a single bit. For example, instead of bit 200₁ being associated only with Item 2, bit 200₁ may alternatively be associated with Items 2, 3, and 4, meaning that if bit 200₁ is set (e.g., having a value equal to 1) then this indicates that consent has been given to Items 2, 3, and 4.

It is noted that a 24-bit vector is included as an example, and other bit lengths are possible (e.g., 8-bit, 16-bit, 32-bit, 64-bit, 128-bit, and/or the like).

Example 200A of FIG. 2A depicts one potential structure and mapping of a bit vector according to embodiments of the present disclosure. Calling the access function described above (e.g., CM·access (i)) for bit vector 112 as depicted in example 200A, given an input offset value i of 1 would return a value of 0, because the value of bit 210₁ is 0.

Example 200B of FIG. 2B depicts example results of the count one and count zero functions described above when applied to bit vector 112. As depicted, calling the count one function described above (e.g., CM·count₁(i)) for bit vector 112 as depicted in example 200B, given an input offset value i of 10 would return a value of 5, because there are 5 one values up to, but not including bit 210₁₀ (e.g., bits 210₀, 210₂, 210₃, 210₆, and 210₇ are set). Furthermore, as depicted, calling the count zero function described above (e.g., CM·count₀(i)) for bit vector 112 as depicted in example 200B, given an input offset value i of 16 would return a value of 6, because there are 6 zero values up to, but not including bit 210₁₆ (e.g., bits 210₁, 210₄, 210₅, 210₈, 210₉, and 210₁₂ are not set).

Example 200C of FIG. 2C depicts example results of the select one and select zero functions described above when applied to bit vector 112. As depicted, calling the select one function described above (e.g., CM·select₁(i)) for bit vector 112 as depicted in example 200C, given an input count value i of 4 would return a value of 7, because bit 210₇ (e.g., at offset 7) is the maximum offset for which the count of ones up to, but not including that offset is equal to 4 (e.g., bits 210₀, 210₂, 210₃, and 210₆). Furthermore, as depicted, calling the select zero function described above (e.g., CM·select₀(i)) for bit vector 112 as depicted in example 200C, given an input count value i of 6 would return a value of 16, because bit 210₁₆ (e.g., at offset 16) is the maximum offset for which the count of zeros up to, but not including that offset is equal to 6 (e.g., bits 210₁, 210₄, 210₅, 210₈, 210₉, and 210₁₂ are not set, and the next offset bit that is not set is bit 210₁₆).

The functions depicted and described above may be useful for obtaining consent data from a bit vector based on configured associations (e.g., in a dictionary) between particular items and particular bits and/or bit patterns, For example, a configured association may indicate that if the count of ones up to, but not including, offset 7 is 4 (e.g., this may be referred to as a bit pattern) then this indicates that one or more items (e.g., Item 21) have been consented to. Thus, to determine whether Item 21 has been consented to based on vector CM, a component may call CM·count₁(7) and determine whether the result is equal to 4. Alternatively, a component may call CM·select₁(4) and determine whether the result is equal to 7.

Configured associations may be changed over time without requiring the bit vector data structure or its associated functions to be modified. For example, the bit at offset 20 (e.g., bit 210₂₀) may be changed from being RFU to being associated with Items 21 and 22, and this change may require only an update to a dictionary of configured associations and not any changes to the bit vector data structure or associated functions.

Similar techniques to those described with respect to bit vector 112 may also be used to obtain data from bit vectors storing other information, such as metadata associated with consent information.

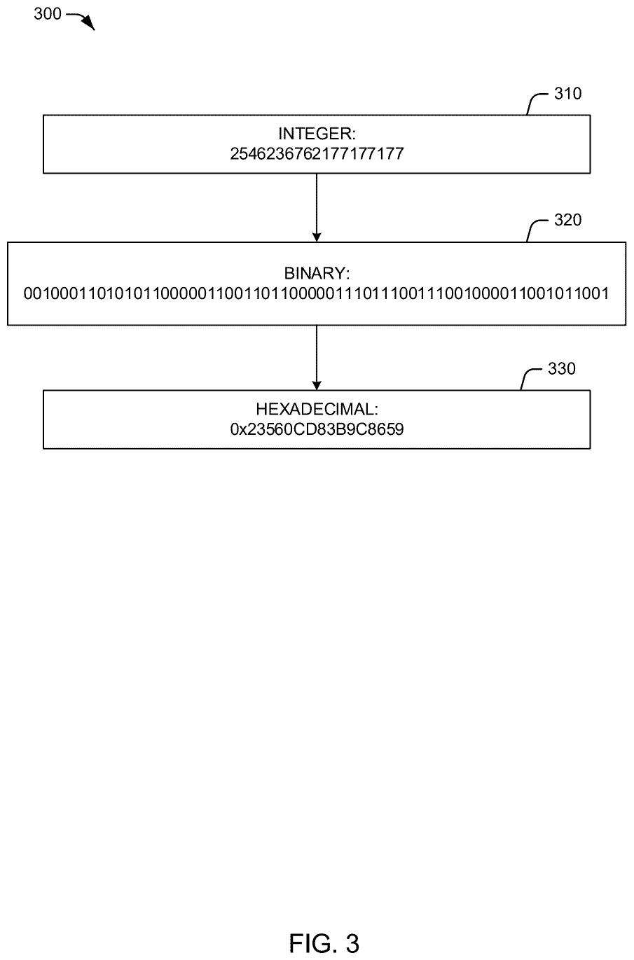

FIG. 3 illustrates an example 300 of storage formats for resource-efficient consent management, in accordance with certain embodiments of the present disclosure.

Example 300 demonstrates that an integer 310 may be represented in the form of binary 320 or other forms such as hexadecimal 330. For instance, the integer 310 value of 2546236762177177177 may be represented as a 64-bit integer in the form of binary 320 value of 0010001101010110000011001101100000111011100111001000011001011001 (e.g., which may be a bit vector used to store consent data as described herein). Additionally, to reduce storage space still further, this bit vector can also be represented in the form of hexadecimal 330 as 0x23560CD83B9C8659. Thus, bit vectors described herein may be further compressed by being stored in hexadecimal form.

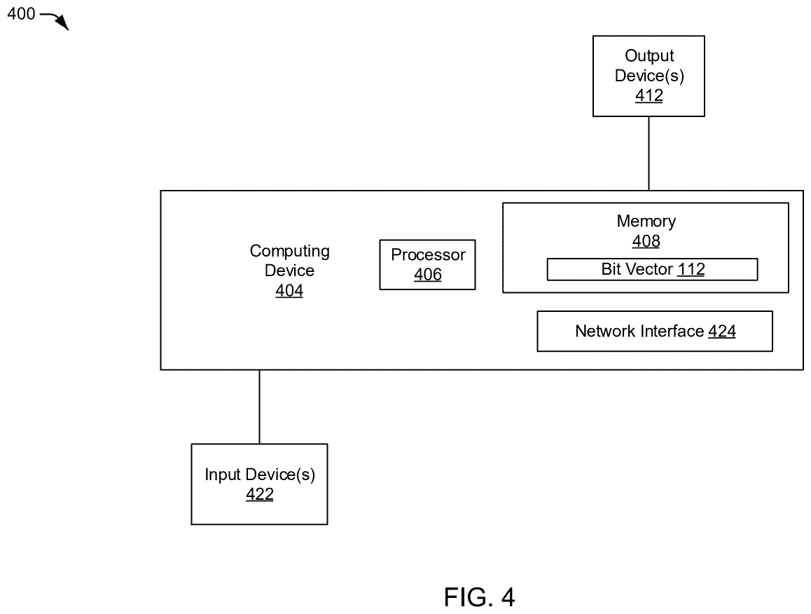

FIG. 4 illustrates an example 400 of a computing device 404 for resource-efficient consent management, in accordance with certain embodiments of the present disclosure. For example, computing device 404 may be representative of aspects of any of medical device 110, medical device 120, and/or cloud server 130 of FIG. 1.

Computing device 404 may include a processor 406 such as a central processing unit (CPU) and a memory 408. The processor 406 can retrieve and store application data in the memory 408, as well as retrieve and execute instructions stored in the memory 408. An interconnect may transmit programming instructions and application data among the processor 406, the memory 408, the network interface 424, the input device(s) 422, and/or the output device(s) 412. The processor 406 can represent a single CPU, multiple CPUs, a single CPU having multiple processing cores, and/or the like. The memory 408 represents volatile memory such as random access memory and/or persistent storage such as a disk drive. Although shown as a single unit, memory 408 can be a combination of fixed or removable storage devices, such as fixed disc drives, removable memory cards or optical storage, network attached storage (NAS), or a storage area-network (SAN).

Computing device 404 can include a network interface 424 for connection with a data communications network, such as to communicate with other devices. The data communications network can be, or can include, one or more of a private network, a public network, a local or wide area network, the Internet, combinations of the same, and/or the like. The data communications network can include, for example, interfaces (e.g., application programming interfaces) for enabling interaction and communication between and among the components and systems of the computing environment (e.g., of FIG. 1) and/or other components and systems.

Computing device 404 may be further associated with one or more input devices 422, which may include one or more components such as a mouse, keyboard, touch device, microphone, camera, button, and/or the like. Computing device 404 may be further associated with one or more output devices 412, which may include one or more components such as a display (e.g., monitor or screen), speaker, light, and/or the like.

The memory 408 can include bit vector 112 of FIGS. 1 and 2A, 2B, and 2C. Furthermore, while not shown, the memory 408 may further include one or more additional bit vectors, one or more event loops, one or more configured associations (e.g., in a dictionary), one or more consent management application and/or components, and/or one or more other types of logic and/or data related to techniques described herein.

It is noted that computing device 404 is included as an example, and techniques described herein may be implemented via fewer or more components, either on the same or different devices, and devices may include physical and/or virtual devices.

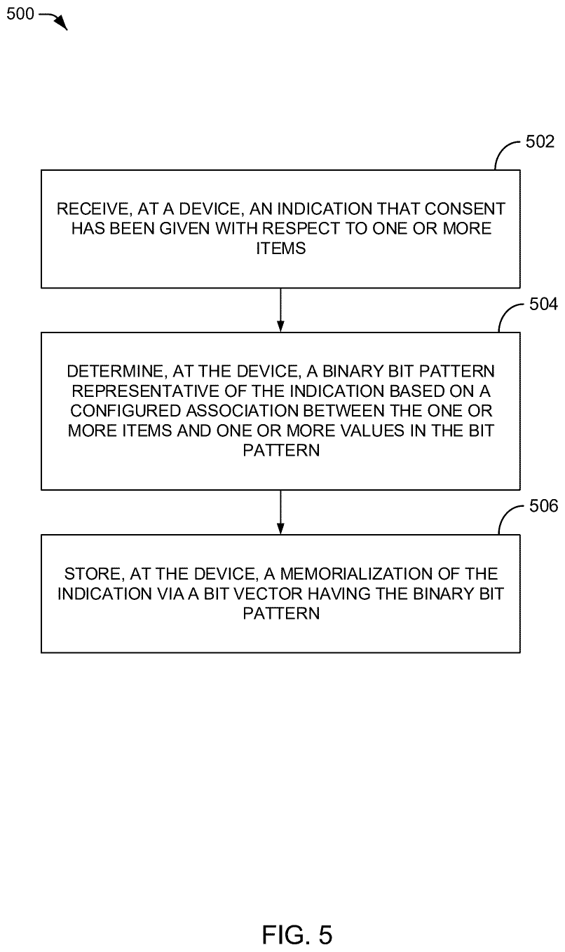

FIG. 5 illustrates an example of a process 500 for resource-efficient consent management, in accordance with certain embodiments of the present disclosure. In certain embodiments, the process 500 can be implemented by one or more components described above with respect to FIG. 1 and/or FIG. 4, and may involve one or more bit vectors and/or functions such as those described above with respect to FIGS. 2A, 2B, 2C, and/or FIG. 3. It is noted that any number of systems, in whole or in part, can implement the process 500.

Process 500 begins at block 502, with receiving, at a device, an indication that consent has been given with respect to one or more items.

Process 500 continues at block 504, with determining, at the device, a binary bit pattern representative of the indication based on a configured association between the one or more items and one or more values in the binary bit pattern.

In some embodiments, the determining of the binary bit pattern comprises setting one or more particular digits in the binary bit pattern to a value of one based on associations between the one or more items and the one or more particular digits.

In certain embodiments, the one or more items comprise a plurality of items, and wherein the determining of the binary bit pattern comprises setting a particular digit in the binary bit pattern to a value of one based on an association between the plurality of items and the particular digit.

In some embodiments, the determining of the binary bit pattern comprises setting one or more digits in the binary bit pattern to a value of one or zero based on an association between the one or more items and a count of one values or zero values from a least significant bit in the binary bit pattern up to a given offset in the binary bit pattern.

Process 500 continues at block 506, with storing, at the device, a memorialization of the indication via a bit vector having the binary bit pattern.

Some embodiments further comprise storing, at the device, metadata associated with the indication via an additional bit vector having a corresponding binary bit pattern indicative of the metadata. In certain embodiments, the metadata comprises one or more of: a timestamp; an identifier; or a location.

Some embodiments further comprise transmitting the bit vector to a separate computing device via a network based on an occurrence of a trigger condition.

Certain embodiments further comprise extracting consent information from the bit vector based on: retrieving one or more values of one or more particular digits in the bit vector; retrieving a count of one values from a least significant bit in the bit vector up to, but not including, a particular offset in the bit vector; retrieving a count of zero values from the least significant bit in the bit vector up to, but not including, the particular offset in the bit vector; or retrieving an offset in the bit vector for which a count of one values or zero values from the least significant bit in the bit vector up to, but not including, the offset in the bit vector is equal to a given number.

In some embodiments, the device comprises a medical device configured to perform operations related to one or more medical procedures, and wherein the one or more items relate to the one or more medical procedures.

### EXAMPLE EMBODIMENTS

In certain embodiments, a computer-implemented method of resource-efficient consent management comprises receiving, at a device, an indication that consent has been given with respect to one or more items; determining, at the device, a binary bit pattern representative of the indication based on a configured association between the one or more items and one or more values in the binary bit pattern; and storing, at the device, a memorialization of the indication via a bit vector having the binary bit pattern.

In certain embodiments, a medical device comprises one or more processors; and a memory storing instructions that, when executed by the one or more processors, cause the medical device to receive an indication that consent has been given with respect to one or more items; determine a binary bit pattern representative of the indication based on a configured association between the one or more items and one or more values in the binary bit pattern; and store a memorialization of the indication via a bit vector having the binary bit pattern.

In certain embodiments, a non-transitory computer readable medium storing instructions that, when executed by one or more processors of a device, cause the device to receive an indication that consent has been given with respect to one or more items; determine a binary bit pattern representative of the indication based on a configured association between the one or more items and one or more values in the binary bit pattern; and store a memorialization of the indication via a bit vector having the binary bit pattern.

### Additional Considerations

The preceding description is provided to enable any person skilled in the art to practice the various embodiments described herein. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a c c, b-b, b-b-b, b-b-c, c-c, and c-c-c or any other ordering of a, b, and c).

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The various illustrative logical blocks, modules and circuits described in connection with the present disclosure may be implemented or performed with a general purpose processor, a digital signal processor (DSP), an application specific integrated circuit (ASIC), a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

A processing system may be implemented with a bus architecture. The bus may include any number of interconnecting buses and bridges depending on the specific application of the processing system and the overall design constraints. The bus may link together various circuits including a processor, machine-readable media, and input/output devices, among others. A user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further. The processor may be implemented with one or more general-purpose and/or special-purpose processors. Examples include microprocessors, microcontrollers, DSP processors, and other circuitry that can execute software. Those skilled in the art will recognize how best to implement the described functionality for the processing system depending on the particular application and the overall design constraints imposed on the overall system.

If implemented in software, the functions may be stored or transmitted over as one or more instructions or code on a computer-readable medium. Software shall be construed broadly to mean instructions, data, or any combination thereof, whether referred to as software, firmware, middleware, microcode, hardware description language, or otherwise. Computer-readable media include both computer storage media and communication media, such as any medium that facilitates transfer of a computer program from one place to another. The processor may be responsible for managing the bus and general processing, including the execution of software modules stored on the computer-readable storage media. A computer-readable storage medium may be coupled to a processor such that the processor can read information from, and write information to, the storage medium. In the alternative, the storage medium may be integral to the processor. By way of example, the computer-readable media may include a transmission line, a carrier wave modulated by data, and/or a computer readable storage medium with instructions stored thereon separate from the wireless node, all of which may be accessed by the processor through the bus interface. Alternatively, or in addition, the computer-readable media, or any portion thereof, may be integrated into the processor, such as the case may be with cache and/or general register files. Examples of machine-readable storage media may include, by way of example, RAM (Random Access Memory), flash memory, ROM (Read Only Memory), PROM (Programmable Read-Only Memory), EPROM (Erasable Programmable Read-Only Memory), EEPROM (Electrically Erasable Programmable Read-Only Memory), registers, magnetic disks, optical disks, hard drives, or any other suitable storage medium, or any combination thereof. The machine-readable media may be embodied in a computer-program product.

A software module may comprise a single instruction, or many instructions, and may be distributed over several different code segments, among different programs, and across multiple storage media. The computer-readable media may comprise a number of software modules. The software modules include instructions that, when executed by an apparatus such as a processor, cause the processing system to perform various functions. The software modules may include a transmission module and a receiving module. Each software module may reside in a single storage device or be distributed across multiple storage devices. By way of example, a software module may be loaded into RAM from a hard drive when a triggering event occurs. During execution of the software module, the processor may load some of the instructions into cache to increase access speed. One or more cache lines may then be loaded into a general register file for execution by the processor. When referring to the functionality of a software module, it will be understood that such functionality is implemented by the processor when executing instructions from that software module.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112 (f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A computer-implemented method of resource-efficient consent management, the method comprising:
receiving, at a device, an indication that consent has been given with respect to one or more items;
determining, at the device, a binary bit pattern representative of the indication based on a configured association between the one or more items and one or more values in the binary bit pattern; and
storing, at the device, a memorialization of the indication via a bit vector having the binary bit pattern.

2. The computer-implemented method of claim 1, wherein the determining of the binary bit pattern comprises setting one or more particular digits in the binary bit pattern to a value of one based on associations between the one or more items and the one or more particular digits.

3. The computer-implemented method of claim 1, wherein the one or more items comprise a plurality of items, and wherein the determining of the binary bit pattern comprises setting a particular digit in the binary bit pattern to a value of one based on an association between the plurality of items and the particular digit.

4. The computer-implemented method of claim 1, wherein the determining of the binary bit pattern comprises setting one or more digits in the binary bit pattern to a value of one or zero based on an association between the one or more items and a count of one values or zero values from a least significant bit in the binary bit pattern up to a given offset in the binary bit pattern.

5. The computer-implemented method of claim 1, further comprising storing, at the device, metadata associated with the indication via an additional bit vector having a corresponding binary bit pattern indicative of the metadata.

6. The computer-implemented method of claim 5, wherein the metadata comprises one or more of: a timestamp; an identifier; or a location.

7. The computer-implemented method of claim 1, further comprising transmitting the bit vector to a separate computing device via a network based on an occurrence of a trigger condition.

8. The computer-implemented method of claim 1, further comprising extracting consent information from the bit vector based on:
retrieving one or more values of one or more particular digits in the bit vector;
retrieving a count of one values from a least significant bit in the bit vector up to, but not including, a particular offset in the bit vector;
retrieving a count of zero values from the least significant bit in the bit vector up to, but not including, the particular offset in the bit vector; or
retrieving an offset in the bit vector for which a count of one values or zero values from the least significant bit in the bit vector up to, but not including, the offset in the bit vector is equal to a given number.

9. The computer-implemented method of claim 1, wherein the device comprises a medical device configured to perform operations related to one or more medical procedures, and wherein the one or more items relate to the one or more medical procedures.

10. A medical device, comprising:
one or more processors; and
a memory storing instructions that, when executed by the one or more processors, cause the medical device to:
receive an indication that consent has been given with respect to one or more items;
determine a binary bit pattern representative of the indication based on a configured association between the one or more items and one or more values in the binary bit pattern; and
store a memorialization of the indication via a bit vector having the binary bit pattern.

11. The medical device of claim 10, wherein to determine the binary bit pattern comprises setting one or more particular digits in the binary bit pattern to a value of one based on associations between the one or more items and the one or more particular digits.

12. The medical device of claim 10, wherein the one or more items comprise a plurality of items, and wherein to determine the binary bit pattern comprises setting a particular digit in the binary bit pattern to a value of one based on an association between the plurality of items and the particular digit.

13. The medical device of claim 10, wherein to determine the binary bit pattern comprises setting one or more digits in the binary bit pattern to a value of one or zero based on an association between the one or more items and a count of one values or zero values from a least significant bit in the binary bit pattern up to a given offset in the binary bit pattern.

14. The medical device of claim 10, wherein the instructions, when executed by the one or more processors, further cause the medical device to store metadata associated with the indication via an additional bit vector having a corresponding binary bit pattern indicative of the metadata.

15. The medical device of claim 14, wherein the metadata comprises one or more of: a timestamp; an identifier; or a location.

16. The medical device of claim 10, wherein the instructions, when executed by the one or more processors, further cause the medical device to transmit the bit vector to a separate computing device via a network based on an occurrence of a trigger condition.

17. The medical device of claim 10, wherein the instructions, when executed by the one or more processors, further cause the medical device to extract consent information from the bit vector based on:
retrieving one or more values of one or more particular digits in the bit vector;
retrieving a count of one values from a least significant bit in the bit vector up to, but not including, a particular offset in the bit vector;
retrieving a count of zero values from the least significant bit in the bit vector up to, but not including, the particular offset in the bit vector; or
retrieving an offset in the bit vector for which a count of one values or zero values from the least significant bit in the bit vector up to, but not including, the offset in the bit vector is equal to a given number.

18. The medical device of claim 10, wherein the medical device is configured to perform operations related to one or more medical procedures, and wherein the one or more items relate to the one or more medical procedures.

19. A non-transitory computer readable medium storing instructions that, when executed by one or more processors of a device, cause the device to:
receive an indication that consent has been given with respect to one or more items;
determine a binary bit pattern representative of the indication based on a configured association between the one or more items and one or more values in the binary bit pattern; and
store a memorialization of the indication via a bit vector having the binary bit pattern.

20. The non-transitory computer readable medium of claim 19, wherein to determine the binary bit pattern comprises setting one or more particular digits in the binary bit pattern to a value of one based on associations between the one or more items and the one or more particular digits.
